(19) United States
(12) Patent Application Publication
(10) Pub. No.: US 2026/0187587 A1
(43) Pub. Date: Jul. 2, 2026

(54) INVENTORY MANAGEMENT SYSTEM

(71) Applicant: Intrinsiq Specialty Solutions, Inc., Carrollton, TX (US)

(72) Inventors: William Jones, Pomfret Center, CT (US); Andrew Allen, Richardson, TX (US); Nicholas Lewis, Midland City, AL (US); Kalpana Kowkutla, Allen, TX (US); Brook Kailey, Mount Vernon, TX (US); Zac Yarbrough, Headland, AL (US); Lizzy Villere, Whitehouse, TX (US); Amit Kumar Diwan, Little Elm, TX (US); Kavita Bhargava, Allen, TX (US)

(21) Appl. No.: 19/431,330

(22) Filed: Dec. 23, 2025

Related U.S. Application Data
(60) Provisional application No. 63/738,922, filed on Dec. 26, 2024.

(51) Int. Cl. G06Q 10/087; G06Q 30/0201; G06Q 30/0283; G16H 20/10

(52) U.S. Cl. CPC G06Q 10/08726; G06Q 10/08741; G06Q 30/0206; G06Q 30/0283; G16H 20/10

(57) ABSTRACT

A method of managing inventory is provided. The method includes receiving information from an end user via a computer-based or web-based inventory management software application having least waste logic. The information includes an amount of a dose of a medication required by a patient treatment plan. The method also includes automatically determining with the inventory management software application and displaying to the end user a recommended combination of vials selected from vials of different sizes to fulfill the dose according to a least waste, least cost, or both basis. Electronic devices and an inventory management system are also disclosed.

### CROSS REFERENCE TO RELATED APPLICATIONS

This application claims the benefit under 35 USC § 119(e) of U.S. Provisional Patent Application No. 63/738,922 filed Dec. 26, 2024.

### BACKGROUND

The present disclosure relates to inventory management systems, for instance, such as may be used in healthcare environments including clinics, specialty physician practices, and the like.

By way of example, inventory management systems may be used in healthcare environments to procure medication and to administer the inventory and distribution of medication and other medical supplies for patients. Accordingly, management systems aid healthcare practices in optimizing inventory based on scheduled patient treatments when purchasing and matching on-hand inventory with a patient's treatment plan (“medication order”) at time of treatment. This functionality may be provided by reviewing which items are part of a treatment plan, comparing this information with current on-hand inventory, and suggesting total dosage that a clinic should purchase and dispense. The goal is to help practices ensure they purchase and dispense the correct quantity of medications for their clinic's planned treatments.

### SUMMARY

According to an embodiment, a method of managing inventory is provided. The method includes receiving information from an end user via a computer-based or web-based inventory management software application having least waste logic, the information including an amount of a dose of a medication required by a patient treatment plan. The method includes automatically determining with the inventory management software application and displaying to the end user a recommended combination of vials selected from vials of different sizes to fulfill the dose according to a least waste, least cost, or combination least waste and least cost basis. The method can also include ordering for purchase the recommended combination of vials or dispensing from inventory the recommended combination of vials.

According to another embodiment, an electronic device is provided. An electronic device comprising at least one non-transitory computer readable storage medium having inventory management computer program instructions with least waste logic stored thereon that, when executed by at least one processor, causes the at least one processor to receive information from an end user entered via of an interface, the information including an amount of a dose of a medication required by a patient treatment plan, and to determine and display to the end user a recommended combination of vials selected from vials of different sizes to fulfill the dose according to a least waste, least cost, or both basis. By way of example, the electronic device may be a computer or remote or cloud server.

According to a further embodiment, an inventory management system is provided. The inventory management system includes a storage cabinet in association with a computer-based or web-based inventory management software application. The storage cabinet stores an inventory of medications, and the computer-based or web-based inventory management software application is configured to provide a recommended combination of vials selected from vials of different sizes to fulfill a dose required by a treatment plan according to least waste, least cost, or both.

### BRIEF DESCRIPTION OF THE DRAWINGS

The foregoing and other objects, features and advantages of the embodiments disclosed herein should become apparent from the following description when taken in conjunction with the accompanying drawings.

FIG. 1 is a chart providing information with respect to vial sizes available for different medicines and combination of vials that will provide the least amount of waste according to an embodiment.

FIG. 2 is a screen view of a display of an inventory management system provided during a process of ordering medications according to an embodiment.

FIG. 3 is a screen view of a display of an inventory management system when requested to configure a combination of vial sizes to fulfill an order according to an embodiment.

FIG. 4 is a screen view of a display of an inventory management system provided during a process of reviewing medications in inventory according to an embodiment.

FIG. 5 is a screen view of a display of an inventory management system during a process of dispensing a medication from inventory according to an embodiment.

FIG. 6 is a screen view of a display of an inventory management system when requested to configure a combination of vial sizes to fulfill a treatment from inventory according to an embodiment.

FIG. 7 is a screen view of a display of an inventory management system similar to FIG. 6, except with one of the options removed, to configure a combination of vial sizes to fulfill a treatment from inventory according to an embodiment.

FIG. 8 is a screen view of a display of an inventory management system provided during a process of ordering or dispensing medications according to an embodiment.

FIG. 9 is a screen view of a display of an unconfigured Med Order shown in FIG. selected for configuration.

FIG. 10 is a screen view of a display of a suggested configuration of vials to fulfill a dose;

FIG. 11 is a screen view of a display of the configured Med Order;

FIG. 12 is a screen view of a display of a configured Med Order that is to be re-configured;

FIG. 13 is a screen view of a display of a partially configured Med Order;

FIG. 14 is a screen view for requesting re-configuration of the partially configured Med Order;

FIG. 15 is a screen view of a display of a suggested configuration of vials to fulfill a dose;

FIG. 16 is a screen view of a display of a suggested re-configuration of vials to fulfill a dose;

FIG. 17 is a screen view of a display of the re-configured Med Order;

FIG. 18 is a screen view of an inventory of items;

FIG. 19 is a screen view of an item selected from inventory;

FIG. 20 is a screen view similar to FIG. 19 with an amount to be dispensed entered;

FIG. 21 is a screen view of a display of suggested configuration of vials to fulfill the entered amount to be dispensed from inventory;

FIG. 22 is a screen view of the inventory of items with the vials selected to fulfill a dose indicated; and

FIG. 23 is a perspective view of an inventory management system including a storage cabinet according to an embodiment.

### DETAILED DESCRIPTION

For simplicity and illustrative purposes, the principles of the embodiments are described by referring mainly to examples thereof. In the following description, numerous specific details are set forth in order to provide a thorough understanding of the embodiments. It will be apparent however, to one of ordinary skill in the art, that the embodiments may be practiced without limitation to these specific details. In some instances, well known devices, apparatus, methods and structures have not been described in detail so as not to unnecessarily obscure the embodiments.

According to embodiments disclosed herein, an inventory management system is provided that assists practices and clinics to accurately track and manage product, such as medicines or drugs. The system may include a computer-based or web-based software application that may allow a user (i.e., authorized personnel of a practice or clinic) to view inventory levels, adjust inventory levels based on products dispensed, and purchase a proper supply to refill inventory based on expected needs. The software application may work in concert with secure dispensing stations/cabinets at the practice and may automatically optimize waste calculations and record data to stored patient histories. The system may also enable critical, cost-saving reports to be produced, simplify clinical workflows, and streamline purchase order processing.

According to an embodiment, purchasing and dispensing of medications may be optimized based on scheduled patient treatments compared with on-hand inventory for a patient's treatment plan (“medication order”). This functionality may be provided by reviewing which items are part of a treatment plan, comparing this information to current on-hand inventory, and suggesting total dosage that a clinic should purchase and dispense. In addition, further functionality includes factoring in the size and cost of each vial of medicine to aid practices not only in ensuring that they order the correct needed amounts but also to optimize each patient treatment based on product availability and cost.

By way of example, for a particular drug that may be required to be dispensed for treatment of a patient or patients, the drug may be provided in different vial sizes, such as two different vial sizes available for purchase, 150 mg and 600 mg. Each patient may have a specific amount of this drug scheduled within their planned treatment, with clinics potentially treating several different patients on a given day. The functionality of the system may sum the total dosage for scheduled patient treatments and suggest purchasing quantities.

According to an embodiment, the inventory management system may also factor in additional information, such as cost and availability of different vial sizes, when suggesting purchases of the drug. Thus, the purchasing and inventory functions of the inventory management system may include so-called least waste logic which automatically factors in cost and availability of different vial sizes, across all the medications managed and all scheduled patient treatments.

By way of specific example, the drugs Leucovorin and Opdivo may vary in vial sizes available for purchase by different practices/clinics and may be at different costs for each practice based on different contract pricing. One practice may be able to purchase more or less vial sizes than another practice at different pricing so that least waste and cost calculations and recommendations by the inventory management system may differ. Each practice has different contract pricing that is a factor in making purchasing recommendations such that each recommendation is necessarily unique.

The benefits for the practice/clinic provided by use of the least waste logic may include costs savings, improved ordering accuracy, and time savings. For instance, the amount of waste primarily when using single dose vials (SDVs) can be minimized to decrease and optimize spend. Further, different vial sizes can vary significantly in price thereby impacting the efficiency of product usage. By way of example, a 120 mg vial may be selected for purchase instead of a 90 mg vial because the 90 mg may actually be more expensive for a particular contract pricing schedule. The least waste logic may also provide demand-based ordering suggestions that account for both best price and best vial size combination. When the appropriate ordering suggestions are considered, then the least waste in a patient's treatment will therefore also be maximized. Also, near real-time visibility/notifications into new product sizing and pricing (availability or discontinued, shortages, etc.) may also be considered by the inventory management system so that accurate updated information is used by the system and made available to the purchaser at the time of purchase.

The benefits for the staff of the practice/clinic provided by use of the above referenced least waste logic may include a reduction in the amount of clicks a nurse may be required to make when reviewing and selecting combinations of vials for a patient's treatment, and improved treatment accuracy (less mental math on a human calculating least waste) directly impacting patient safety.

The benefits for the patient of the practice/clinic provided by use of the above referenced least waste logic may include optimizing vial size combinations per medication and per patient that also considers current on-hand inventory (assigned and in stock) while also recommending least waste alternatives. Finally, benefits for payers/insurance billing may include the elimination of excessive waste purchased which is typically frowned upon when claims are forwarded and reviewed especially in a scenario where a different combination of vial sizes could have been used to minimize waste for a patient's treatment.

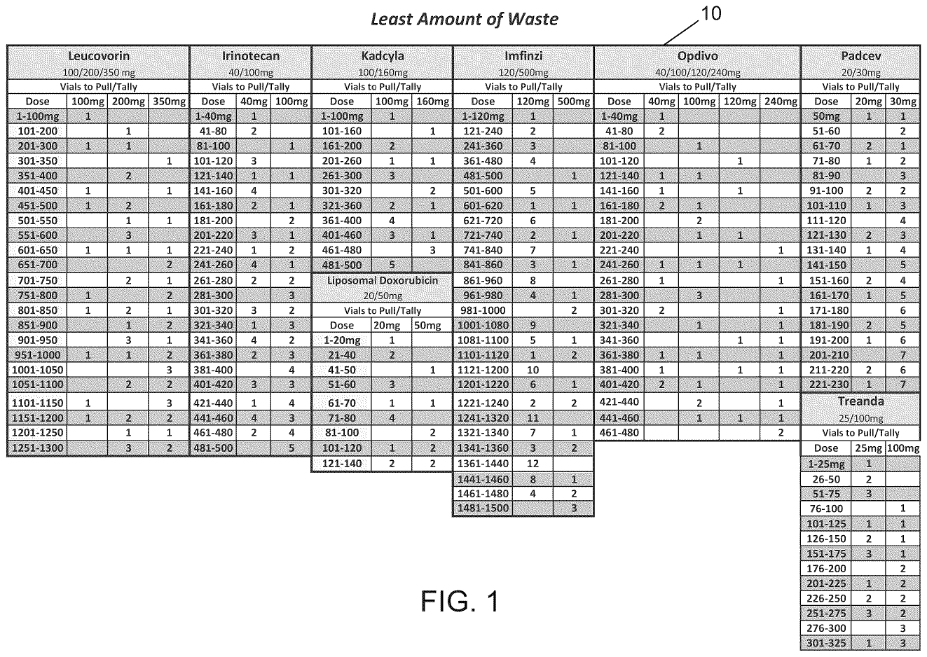

FIG. 1 provides an example of a chart 10 relating to least amount of waste considerations for a particular practice. For example, several different medications are listed with each being available in inventory and/or for purchase in different sizes of vials. Accordingly, depending upon the dose required for patient treatment, the chart indicates the combination of different sized vials that will provide the necessary dose with the least amount of waste upon use. In this particular example for purposes of ease of explanation, cost is not considered.

As an example, if 250 mg of Opdivo is required to be dispensed or ordered, information contained within the chart 10 can be considered which indicates that this medicine is available in four different vial sizes, including 40 mg, 100 mg, 120 mg, and 240 mg. The chart 10 suggests three vials, one each of the sizes 40 mg, 100 mg, and 120 mg. The total amount of the drug in the vials is 260 mg whereby waste is limited to 10 mg.

Such a chart may also consider pricing. For instance, in the above example, it may be less expensive according to a particular contract pricing arrangement to purchase two vials, including one of the 240 mg size and one of the 40 mg size. This would result in waste of 30 mg; however, it may be recommended based on a lower price/cost. Accordingly, the management system or application would include this information stored for each medication and for each practice and may provide one or both of the above recommendations (one based on least waste and one based on least cost) to the practice for final determination to be made by the practice.

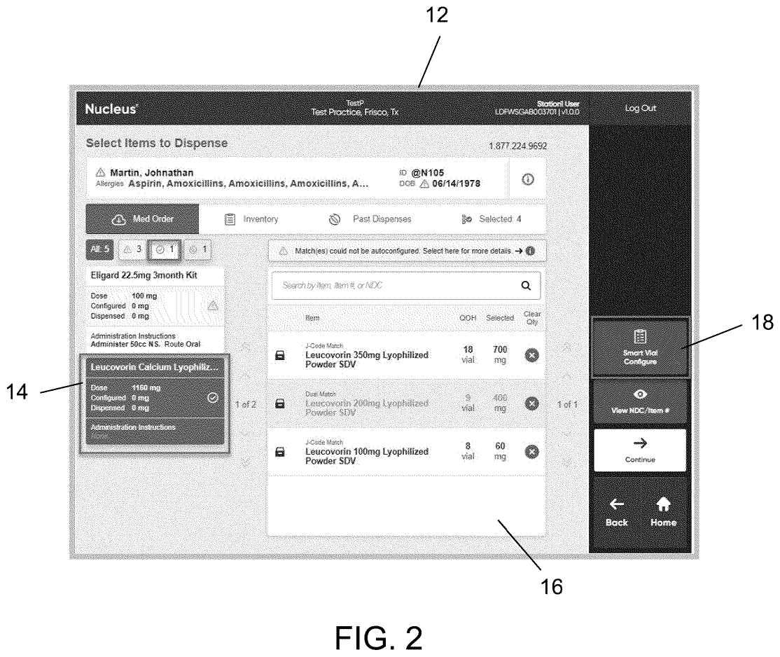

According to another example of an embodiment, FIG. 2 provides a screen shot of a computer or like electronic device that displays a user interface 12 of an inventory management system or application to authorized personnel of a practice or clinic. As shown on the interface 12, the “Med Order” tab 14 has been selected and the display includes a list of drugs/medications required for a particular patient. Within the list of drugs, the drug “Leucovorin Calcium Lypophilized Powder” has been selected (solely for purposes of example). The dose required is indicated as being “1160 mg” for this particular patient and treatment. Accordingly, the management system identifies that the Quantities On-Hand (QOH) (within the practice) is 18 vials of a 350 mg size, 9 vials of a 200 mg size, and 8 vials of a 100 mg size. See section 16 of the display.

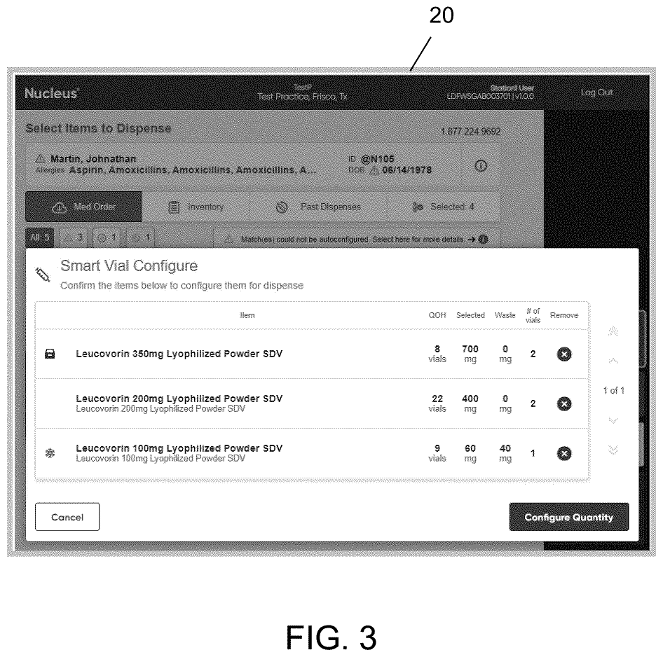

When the “Smart Vial Configure” option (see section 18 of the display) as shown in FIG. 2 is selected, the inventory management application provides a display 20 to the user as appears in FIG. 3. Here, considering costs and sizes of vials, the management system automatically provides a recommendation with respect to a combination of vials to be ordered for purchase to fulfill the requirements of one or more treatment plans. In the example, the least waste logic suggests 2 vials of the 350 mg size, 2 vials of the 200 mg size, and 1 vial of the 100 mg size. This combination limits the waste to 40 mg (i.e., 1200 mg in total within the combination of vials to fulfill the need for a dose of 1160 mg with 40 mg remaining as waste).

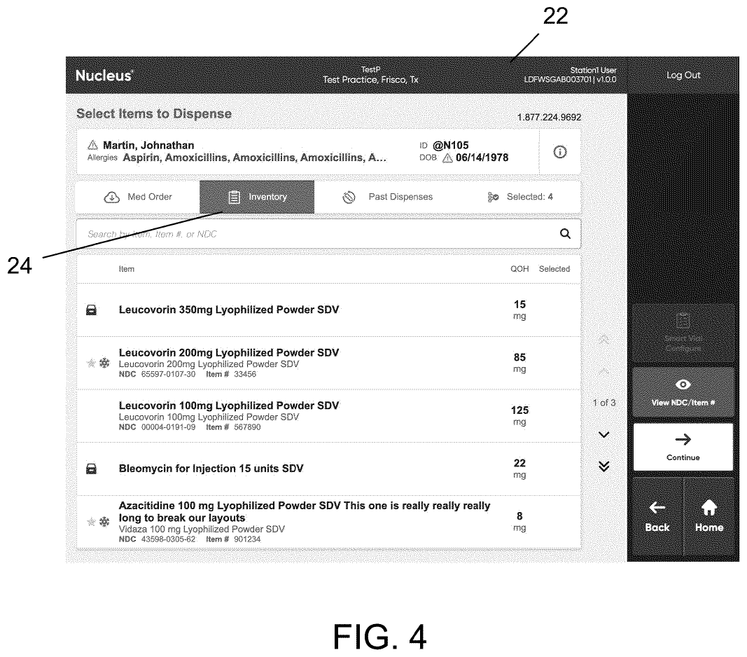

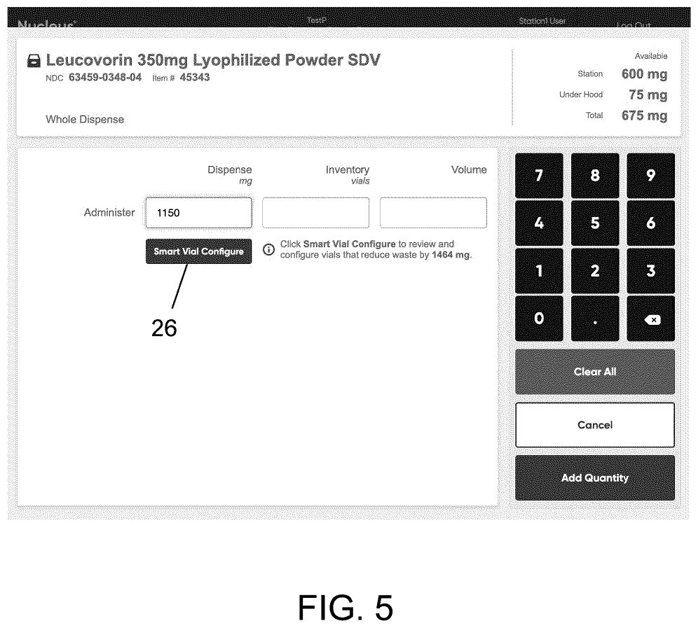

According to a further example of an embodiment, FIG. 4 provides a screen shot of a computer or like electronic device that displays a user interface 22 of an inventory management system or application to authorized personnel of a practice or clinic. As shown on the interface 22, the “Inventory” tab 24 has been selected and the display includes a list of drugs/medications required to be dispensed for a particular patient. Within the list of drugs, the drug “Leucovorin Lypophilized Powder” has been selected (solely for purposes of example) and the dose as entered (see FIG. 5) is indicated as being “1150 mg” for this particular patient and treatment.

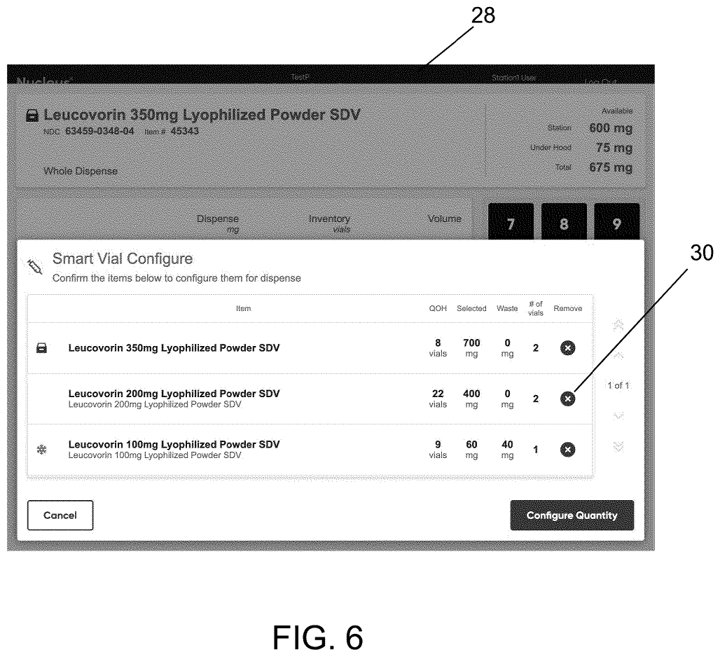

When the “Smart Vial Configure” option (see section 26 of the display) as shown in FIG. 5 is selected, the inventory management application provides a display 28 to the user as appears in FIG. 6. Here, considering costs and sizes of vials, the management system automatically provides a recommendation with respect to a combination of vials to be dispensed from inventory to fulfill the requirements of the treatment plans. In the example, the least waste logic suggests 2 vials of the 350 mg size, 2 vials of the 200 mg size, and 1 vial of the 100 mg size. This combination limits the waste to 40 mg (i.e., 1200 mg in total within the combination of vials to fulfill the need for a dose of 1160 mg with 40 remaining).

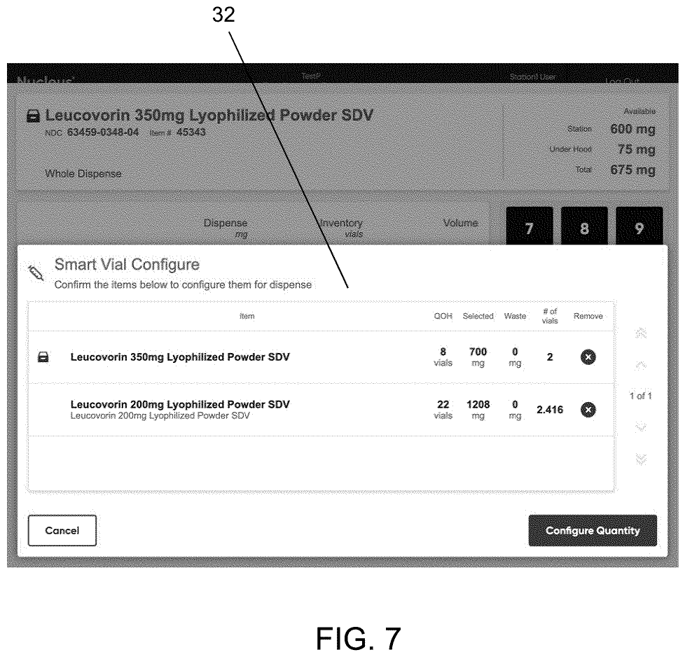

If the user should decide to delete one of the vial size options from the options shown in display 28, the user can select the “remove” button 30 corresponding to the desired option to be deleted. For example, if the 100 mg size is removed from consideration, a display 32 as shown in FIG. 7 is provided. Here, the management system automatically provides a recommendation with respect to a combination of vials of the remaining sizes to be dispensed from inventory to fulfill the requirements of the treatment plans. In the example shown in FIG. 7, the least waste logic suggests 2 vials of the 350 mg size and 3 vials of the 200 mg size. This combination limits the waste to 140 mg (i.e., 1300 mg in total within the combination of vials to fulfill the need for a dose of 1160 mg with 140 remaining).

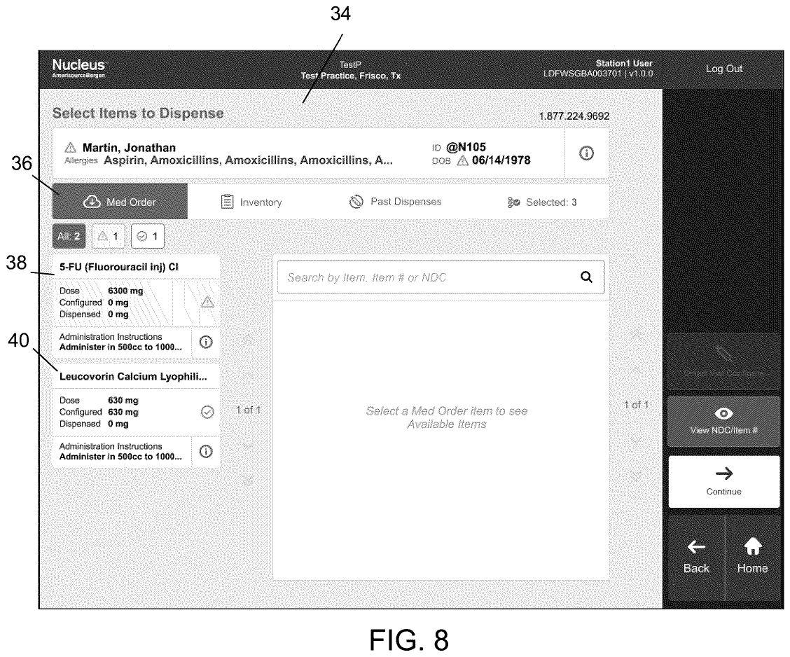

According to a further example of an embodiment, FIG. 8 provides a screen shot of a computer or like electronic device that displays a user interface 34 of an inventory management system or application to authorized personnel of a practice or clinic. As shown on the interface 34, the Med Order tab 36 is selected and the particular patient has two Med Orders, 38 (Fluorouracil) and 40 (Leucoverin Calcium). The Med Order 38 (Fluorouracil) has not been subject to automatic configuration as shown by the caution symbol. Thus, a dose of 6300 mg is indicated; however, 0 mg has been configured. The Med Order 40 (Leucoverin Calcium) has been subject to automatic configuration as shown by the check mark symbol. Thus, a dose of 630 mg is indicated and the 630 mg has been configured by the system.

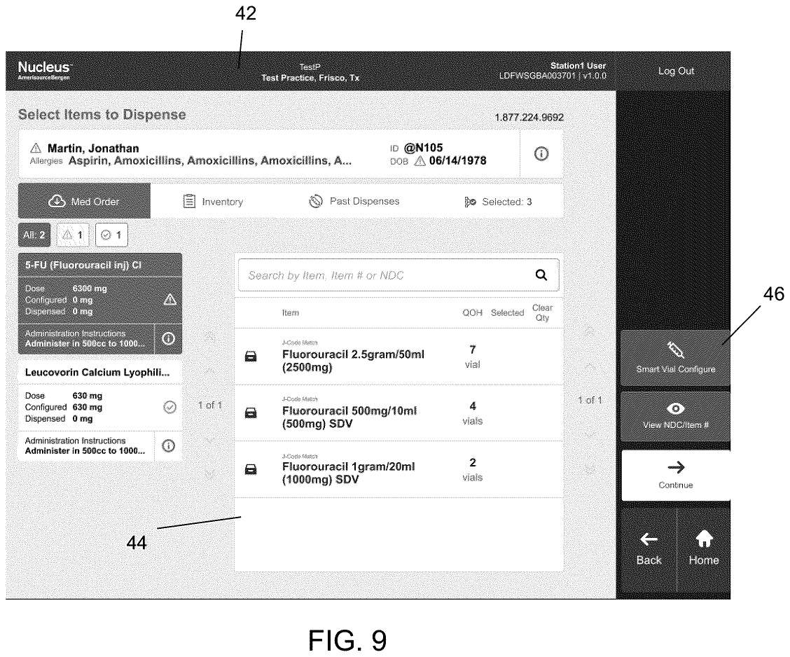

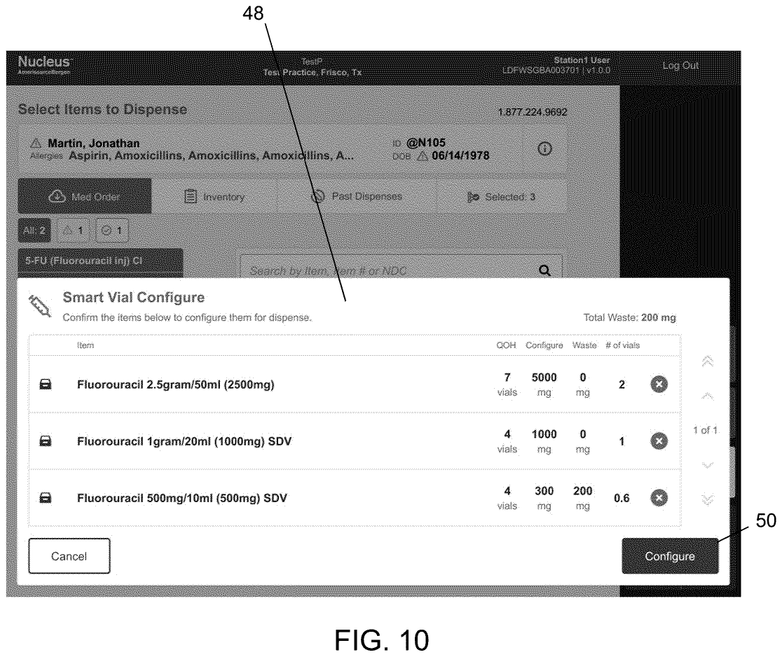

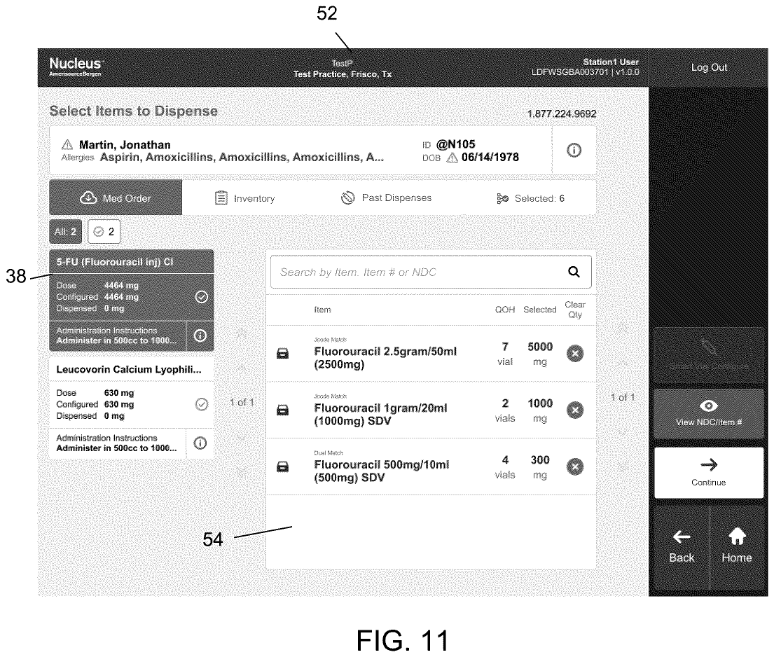

When a user selects the Med Order 38 (Fluorouracil), a display 42 is provided as shown in FIG. 9. The display 42 includes a table 44 that indicates the availability of three different vial sizes for the drug (i.e., 2500 mg, 500 mg and 1000 mg) with none being indicated as “selected”. To configure the dose, a user may select the “Smart Vial Configure” button 46. As a result, a “Smart vial Configuration” display 48 is provided to the user as shown, for instance, in FIG. 10. Here, considering costs and sizes of vials, the management system automatically provides a recommendation with respect to a combination of vials to be ordered for purchase to fulfill the requirements of one or more treatment plans. In the example, the least waste logic initially suggests 2 vials of the 2500 mg size, 1 vial of the 1000 mg size, and 1 vial of the 500 mg size. This combination limits the waste to 200 mg (i.e., 6500 mg in total within the combination of vials to fulfill the need for a dose of 6300 mg with 200 mg remaining as waste). If this configuration is acceptable, the user may hit the “Configure” button 50. After the configure button 50 is selected, the display 52 as shown in FIG. 11 is provided to the user, which shows that the Med Order 38 has been configured/selected and the table 54 on the display 52 has been populated with the “selected” configured items.

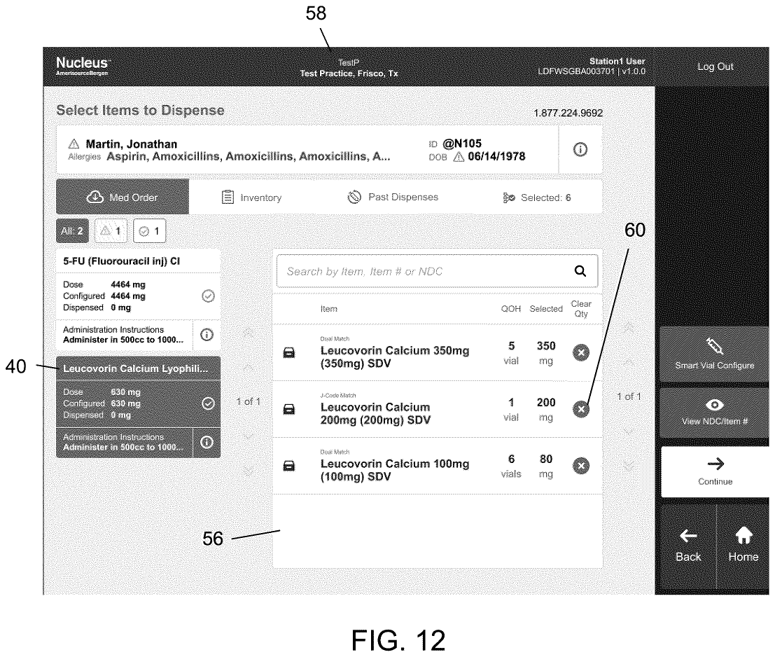

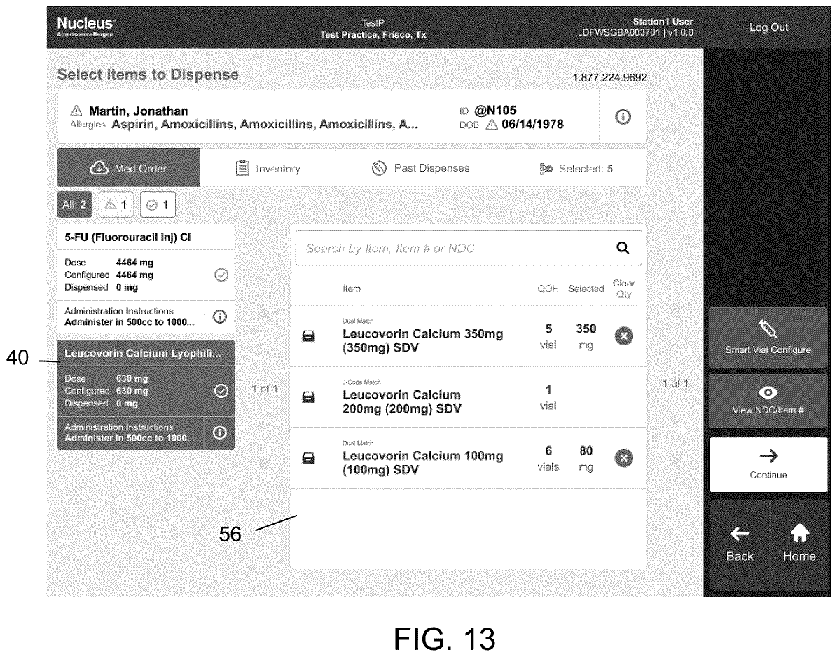

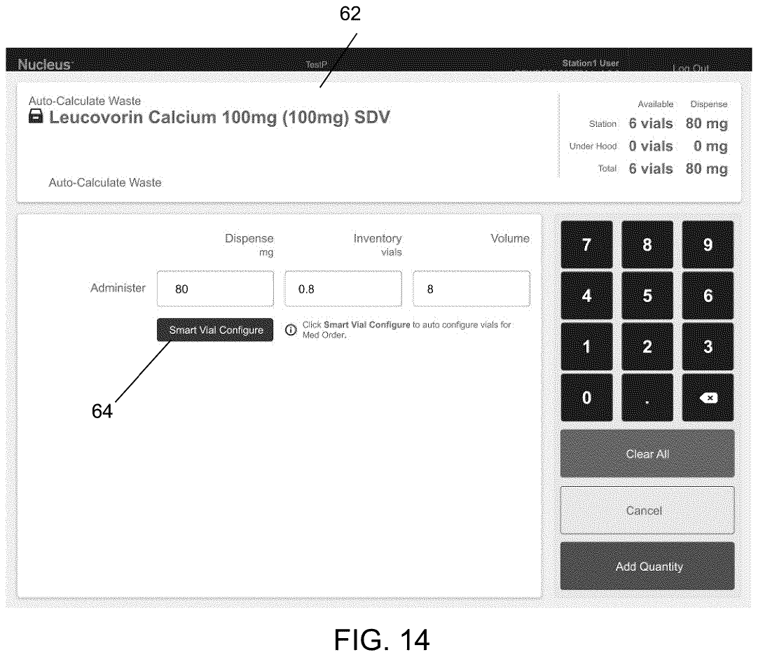

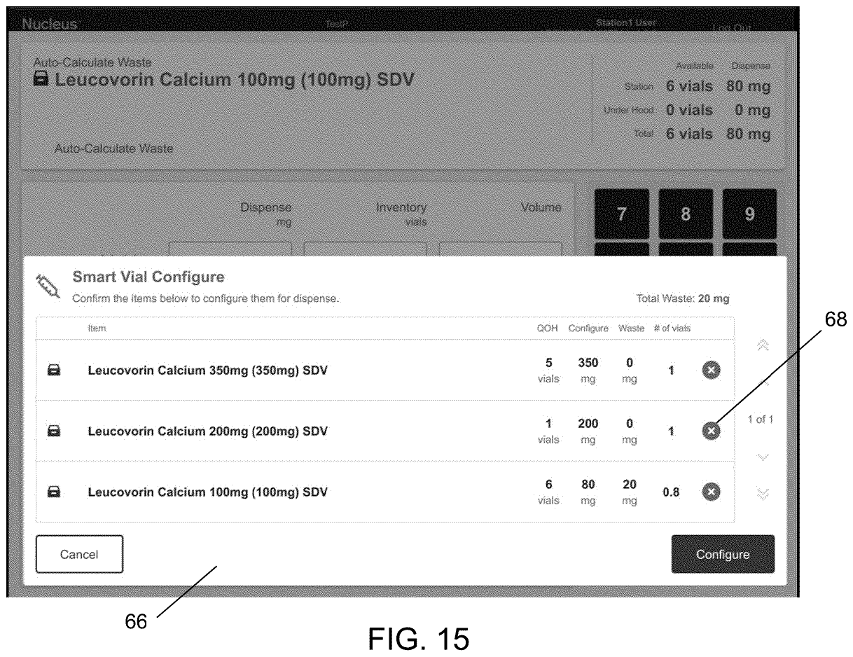

If, for instance, the user decides to re-configure the configuration of the Med Order 40 (Leucovorin Calcium), the user would select the Med Order 40 to have a table 56 of the configured items appear on a display 58, as shown in FIG. 12. If the user decides to remove/clear the selection of the 200 mg vial size vial from the table 56, the user may select the “Clear Quantity” button 60. Accordingly, the Med Order 40 is now only partially configured. For instance, see FIG. 13 which shows only 430 mg (i.e., 350 mg plus 80 mg) of the 630 mg dose has been configured/selected. Thus, if the user desires to configure the remaining requirements of the Med Order 40 with vials selected from the 100 mg and 350 mg sizes, the user may select the 100 mg vials entry in the table 56. This causes a display 62 with an option to select a “Smart Vial Configure” button 64 to appear as shown in FIG. 14. When the button 64 is selected, a display 66 appears as shown in FIG. 15. In this example, the least waste logic initially suggests 1 vial of the 350 mg size, 1 vial of the 200 mg size, and 1 vial of the 100 mg size. This combination limits the waste to 20 mg (i.e., 650 mg in total within the combination of vials to fulfill the need for a dose of 630 mg with 20 mg remaining as waste).

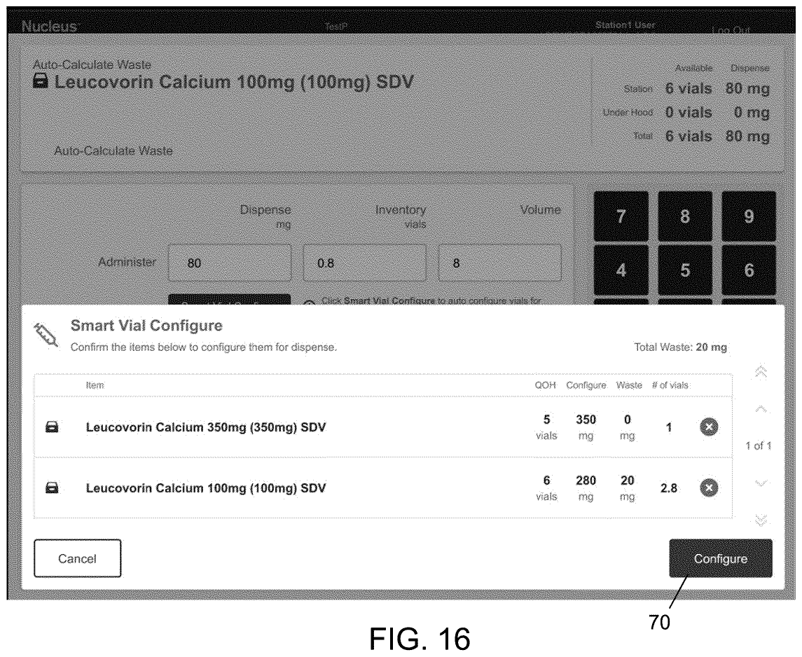

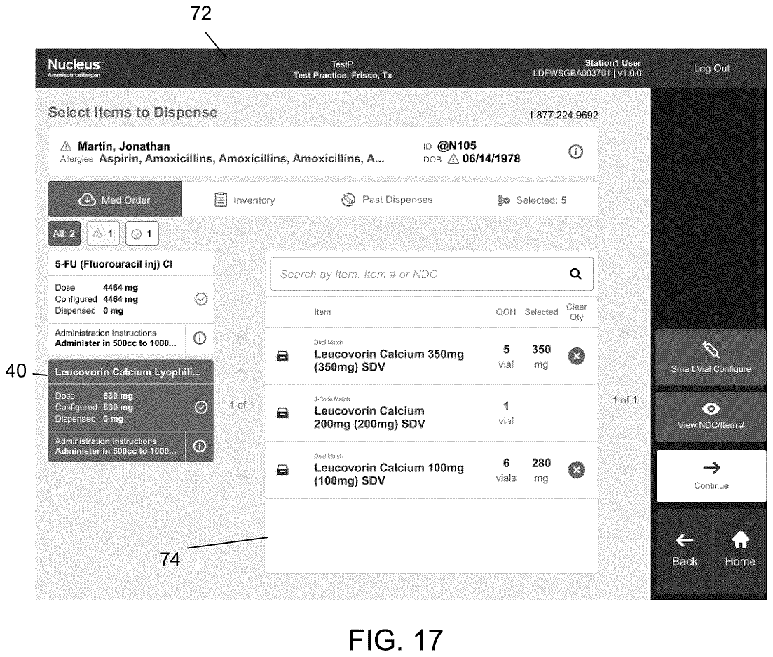

However, in this example, the user may remove consideration of using the 200 mg size vials by selecting the cancel button 68 on display 66. Thereafter, the Smart Vial Configuration updates to a combination of 1 vial of the 350 mg size and 3 vials of the 100 mg size as shown in FIG. 16. This updated combination limits the waste to 20 mg (i.e., 650 mg in total within the combination of vials to fulfill the need for a dose of 630 mg with 20 mg remaining as waste). If this updated configuration is acceptable, the user may hit the “Configure” button 70. After the configure button 70 is selected, the display 72 as shown in FIG. 17 is provided to the user, which shows that the Med Order 40 has been configured and the table 74 on the display has been populated with the selected/configured items.

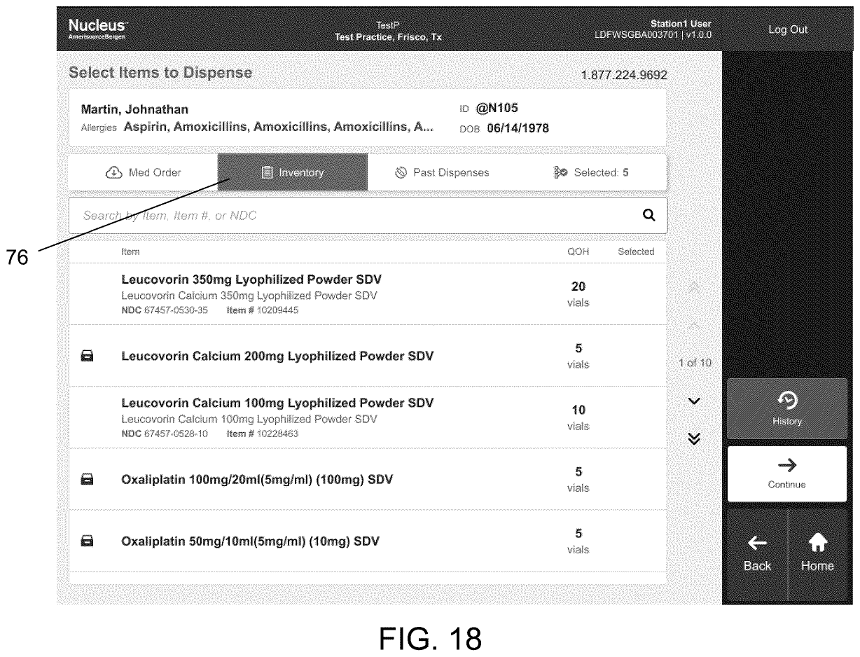

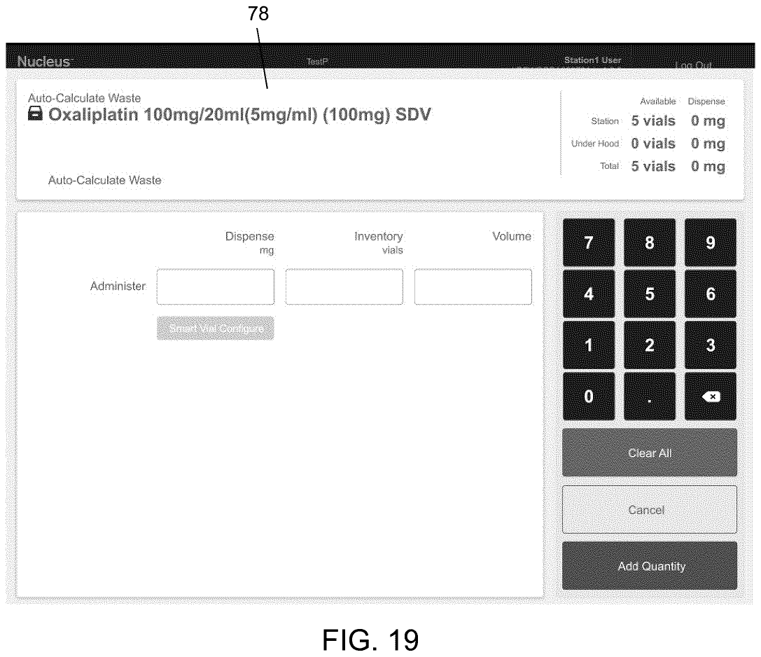

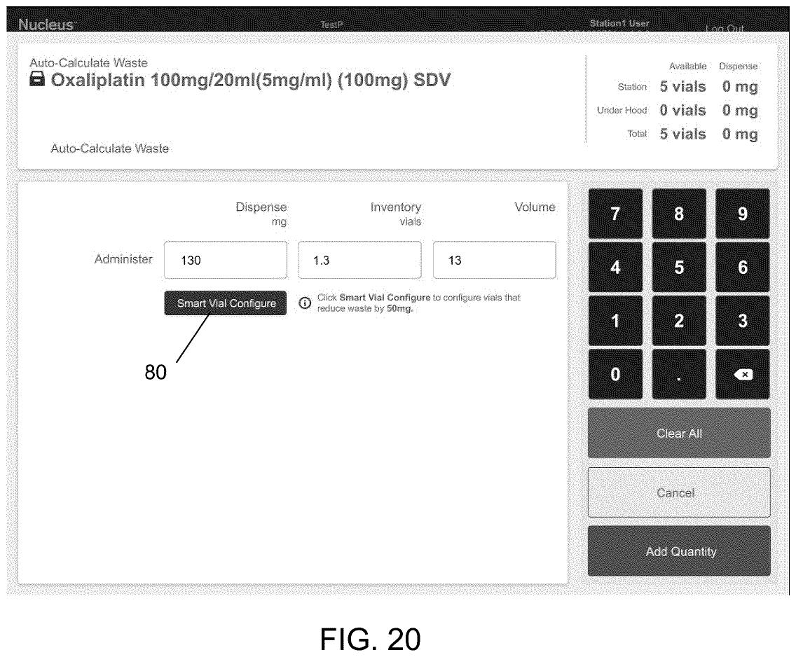

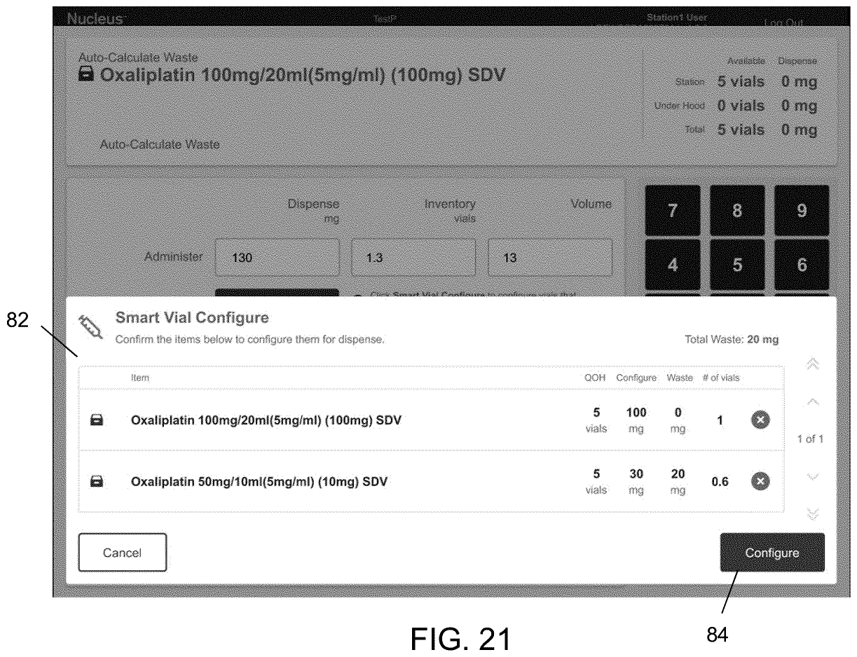

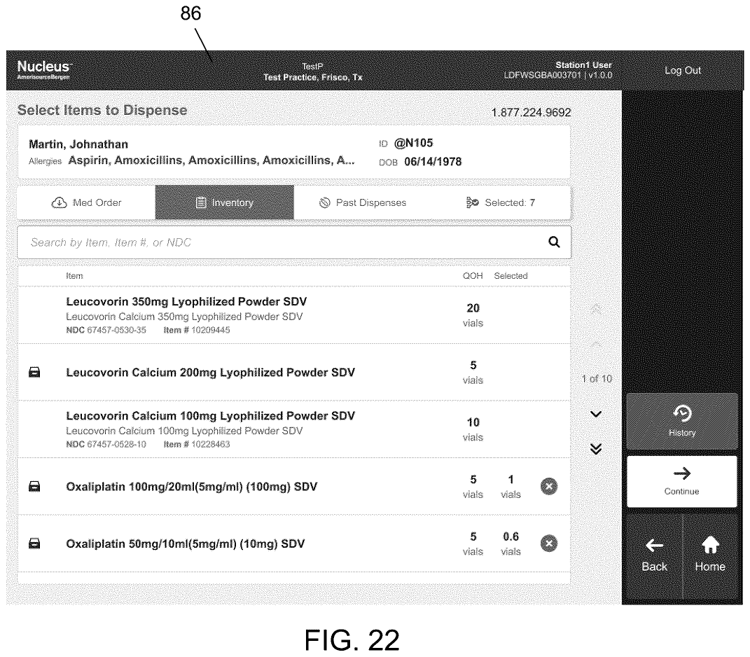

In FIG. 18, the user has selected the “Inventory” tab 76 which provides a listing of all the drugs available in the inventory (i.e., Quantity on Hand). The user may select one of the listed items, such as “Oxaliplatin”, 100 mg size. This results in the display 78 shown in FIG. 19. The user may enter a dosage to dispense, for instance, 130 mg as shown in FIG. 20 and the “Smart Vial Configure” button 80 may be selected. This results in the display 82 shown in FIG. 21 which provides a suggested least waste option for configuration. The suggested configuration includes 1 vial of 100 mg and 1 vial of 50 mg. Thus, the combination totals 150 mg to provide the 130 mg dose with 20 mg of waste. If this is the desired combination, the user may select the “Configure” button 84 which results in the display 86 of FIG. 22 showing that the 100 mg and 50 mg vials of Oxaliplatin have been selected/configured for least waste.

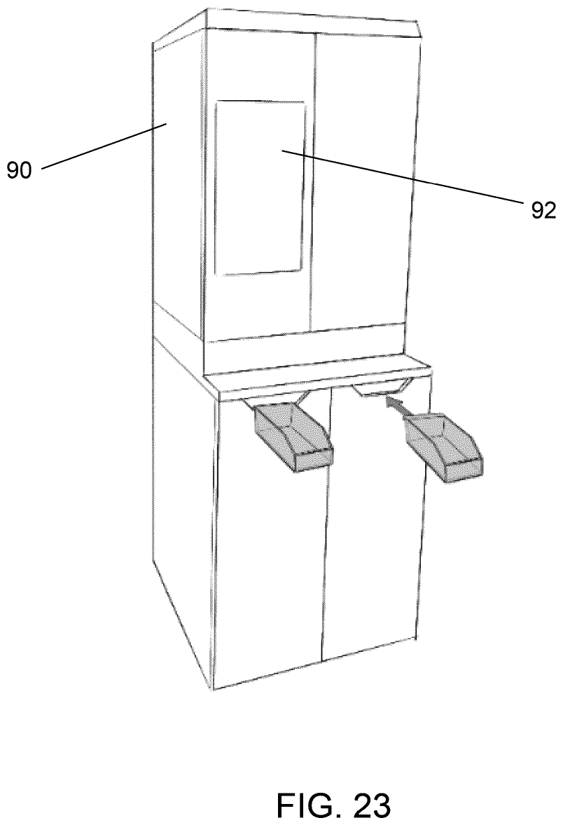

The above referenced software application may be used with a cabinet 90 that together may provide an automated inventory management system. See FIG. 23. A computer operating under the direction of a medication inventory management software application program may control the display of the user interface 92, such as a touch-screen, on or in association with the cabinet 90 and access to and operation of the cabinet 90. For example, the interface may be used to permit access to a particular storage unit, enter patient information, record transactions, update inventory, and the like.

Access to each of the storage units within the cabinet 90 may be limited to properly credentialed healthcare professionals by facility protocols. A healthcare professional may gain access to the medication storage units according to established protocols of the facility. Once access is obtained, the healthcare professional can retrieve medication(s) or other medical supplies from the storage units as they are needed or in order to fulfill the prescription requirements of individual patients which are under the care of the healthcare professional.

The management system may indicate the location of a selected medication or the like stored within the cabinet 90. For example, the medication inventory management software application program may control illumination of lights or the like which is based upon the drug required for a particular patient. Thus, upon entering patient information or drug information for dispensing of a particular medication or the like, the inventory system will cause certain lights to illuminate. Thus, the worker is readily informed of the particular shelf and the particular compartment on the shelf in which the required medication is located.

A system for carrying out any of the above disclosed embodiments, methods, or arrangements may include software or the like provided on a circuit board or within another electronic device such as a remote or cloud service and can include various processors, microprocessors, modules, units, components, controllers, chips, disk drives, and the like. It will be apparent to one of ordinary skill in the art that systems, modules, components, units, processors, servers, and the like may be implemented as electronic components, software, hardware or a combination of hardware and software for purposes of providing a system.

Embodiments may also include at least one non-transitory computer readable storage medium having computer program instructions stored thereon that, when executed by at least one processor, can cause the at least one processor to perform any of the steps described above.

The foregoing description and specific embodiments are merely illustrative of the principles thereof, and various modifications and additions may be made to the apparatus by those skilled in the art, without departing from the spirit and scope of this invention.

We claim:

1. A method of managing inventory, comprising the steps of:
receiving information from an end user via a computer-based or web-based inventory management software application having least waste logic, the information including an amount of a dose of a medication required by a patient treatment plan; and
automatically determining with the inventory management software application and displaying to the end user a recommended combination of vials selected from vials of different sizes to fulfill the dose according to a least waste or least cost basis.

2. The method according to claim 1, further comprising the step of ordering for purchase the recommended combination of vials.

3. The method according to claim 1, further comprising the step of dispensing from inventory the recommended combination of vials.

4. The method according to claim 1, wherein the inventory management software application determines the combination of vials based upon the different sizes of vials available to a particular end user and contract pricing for the particular end user.

5. The method according to claim 1, wherein the least waste logic of the inventory management software application selects the combination of vials such that it fulfills the dose and provides an excess of medication over the amount of the dose as small as possible given the different vial sizes available from inventory or for purchase.

6. The method according to claim 1, wherein an interface provided to the end user by the inventory management software application includes an option to launch the least waste logic and identify the recommended combination of vials.

7. The method according to claim 1, wherein the vials are single dose vials (SDVs).

8. The method according to claim 1, wherein costs of the different vials are determined by the inventory management software application based on contract pricing of the end user and the recommended combination of vials is a combination of vials of least cost that provides the least amount of waste.

9. The method according to claim 1, wherein the inventory management software application is accessed via communications with a remote or cloud-based server.

10. The method according to claim 1, wherein the inventory management software is accessed in association with a cabinet in which inventory is stored.

11. The method according to claim 10, wherein the inventory management software instructs the end user with respect to a location of the vials within the cabinet.

12. An electronic device comprising at least one non-transitory computer readable storage medium having inventory management computer program instructions with least waste logic stored thereon that, when executed by at least one processor, causes the at least one processor to receive information from an end user via of an interface, the information including an amount of a dose of a medication required by a patient treatment plan, and to determine and displaying to the end user a recommended combination of vials selected from vials of different sizes to fulfill the dose according to a least waste or a least cost basis.

13. The electronic device according to claim 12, wherein the at least one non-transitory computer readable storage medium having computer program instructions stored thereon that, when executed by at least one processor, generates an order for purchase of the recommended combination of vials or directs the end user to dispense from on-hand inventory the recommended combination of vials.

14. An inventory management system, comprising a storage cabinet in association with a computer-based or web-based inventory management software application, the storage cabinet storing an inventory of medications, and the computer-based or web-based inventory management software application configured to provide a recommended combination of vials selected from vials of different sizes to fulfill a dose required by a treatment plan according to least waste, least cost, or both.

15. The inventory management system according to claim 14, wherein the inventory management system software application tracks the inventory of medications in the storage cabinet and is configured to generate orders for purchasing the recommended combination of vials and to generate instructions with respect to dispensing the recommended combination of vials from the inventory.

16. The inventory management system according to claim 15, wherein the inventory management software application determines the combination of vials based upon the different sizes of vials available to a particular end user and contract pricing for the particular end user.

17. The inventory management system according to claim 15, wherein the least waste logic of the inventory management software application selects the combination of vials such that it fulfills the dose and provides an excess of medication over the amount of the dose as small as possible given the different vial sizes available from inventory or for purchase.

18. The inventory management system according to claim 15, wherein an interface provided to the end user by the inventory management software application includes an option to launch the least waste logic and identify the recommended combination of vials.

19. The inventory management system according to claim 15, wherein the vials are single dose vials (SDVs), and wherein the inventory management software application instructs the end user with respect to a location of the vials within the storage cabinet.

20. The inventory management system according to claim 15, wherein costs of the different vials are determined by the inventory management software application based on contract pricing of the end user and the recommended combination of vials is a combination of vials of least cost that provides the least amount of waste.
